(19) United States
(12) Patent Application Publication
(10) Pub. No.: US 2010/0266687 A1
(43) Pub. Date: Oct. 21, 2010

(54) IMPROVED TABLET COATING

(75) Inventor: David John Willoughby, Queensland (AU)

Correspondence Address: OCCHIUTI ROHLICEK & TSAO, LLP, 10 FAWCETT STREET, CAMBRIDGE, MA, 02138, US

(73) Assignee: Sanofi-aventis Healthcare Pty Limited, macquarie Park (AU)

(21) Appl. No.: 12/739,460

(22) PCT Filed: Oct. 30, 2008
(86) PCT No.: PCT/AU08/01596

§ 371 Date: Jun. 25, 2010

(30) Foreign Application Priority Data
Nov. 1, 2007 (AU) 2007906008

(51) Int. Cl. A61K 9/36; A61K 47/26; B05D 1/02

(52) U.S. Cl. 424/480; 514/781; 427/2.14

(57) ABSTRACT

The present invention provides a tablet coating composition including a cellulose polymer, a plasticiser, a sweetener, and a powdered flavour composition. The powdered flavour composition includes a flavourant associated with a solid carrier. The present invention also provides a pharmaceutical tablet including a core containing an active agent and a coating formed from the tablet coating composition.

This international patent application claims priority from Australian provisional patent application 2007906008 filed on 1 Nov. 2007, the contents of which are herein incorporated by this reference.

### FIELD

The present invention relates to coatings for tablets for pharmaceutical, nutraceutical and/or veterinary use. More specifically, the present invention relates to flavoured coatings that have a pleasant mouthfeel. The present invention also relates to processes for preparing coated pharmaceutical and/or nutraceutical tablets and/or veterinary tablets.

### BACKGROUND

It is known to provide active agents to individuals for a variety of purposes. Pharmaceutically active agents such as drugs or medicaments can be used to treat diseases or for prophylactic purposes. Nutraceutically active agents can be used for a variety of medical and non-medical purposes including supplementing dietary intake, enhancing performance, and the like.

Oral administration of active agents is the most common mode of administration. In many cases it is desirable to administer active agents as compressed (solid) tablets for oral administration due to reasons of stability, economy, simplicity, and convenience of dosing. Tablets typically deliver a pharmacologically effective amount of an active agent to the gastrointestinal tract of the human or animal to which they are administered.

Tablets can have one or more coatings that provide a variety of benefits, including the masking of objectionable flavours or odors, protecting unstable tablet compositions, improving the ease with which the tablets are swallowed, providing protection of the tablets through the stomach with enteric coatings, improving the appearance of the tablets, improving the mouthfeel of the tablets, colouring the tablets, and the like.

Numerous methods for coating tablets with one or more coatings are known. They include sugar coating, solvent film coating, aqueous film coating, delayed release coating and granule coating techniques.

Some of the most commonly used coatings today are polymeric film coating agents. Advantages of polymeric coatings include the ability to produce a tablet in which the coating comprises less than 5% of the weight, better resistance to chipping, and increased tablet strength. Polymers have been applied to tablets using both aqueous and non-aqueous solvents. Many tablet coatings are formed from low viscosity hydroxypropylmethylcellulose (HPMC) and an appropriate plasticiser. The coating is typically applied by a spraying system or device to a tablet in a coating process.

There is a continual need for tablets in which one or more of the tablet coating properties, such as gloss, mouthfeel, swallowability, palatability, etc is improved. Coating compositions and new processes for preparing such an improved tablet coating economically and efficiently continue to be of interest.

Throughout this specification reference may be made to documents for the purpose of describing various aspects of the invention. However, no admission is made that any reference cited in this specification constitutes prior art. In particular, it will be understood that the reference to any document herein does not constitute an admission that any of these documents forms part of the common general knowledge in the art in any country.

### SUMMARY

The present invention arises from the finding that the use of a powdered flavour composition, which can be obtained, for example, by spray drying a flavourant with a carrier, such as maltodextrin, provides certain manufacturing efficiencies and product benefits for pharmaceutical, nutraceutical and/or veterinary tablets having a flavoured coating. The flavour composition may be used in a tablet coating composition suitable for coating tablets having one or more beneficial properties.

The present invention provides a tablet coating composition including a cellulose polymer, a plasticiser, a sweetener, and a powdered flavour composition, the powdered flavour composition including a flavourant associated with a solid carrier.

The flavourant is “associated with” the solid carrier in that it at least partially coats, is solidified with, absorbed into, or adsorbed onto some of the particles of the carrier in the flavour composition. This can be achieved by spray drying the flavourant with the powdered carrier. As such, in some embodiments, the powdered flavour composition can be said to consist essentially of the flavourant and the carrier.

The carrier may include a dextrin. In some embodiments, the dextrin is maltodextrin.

The powdered flavour composition may also contain other components. For example, the carrier may contain a saccharide, such as glucose. Alternatively, or in addition, the carrier may contain a sweetener, such as a natural or artificial sweetener. Alternatively, or in addition, the carrier may contain a gum, such as sodium carboxymethylcellulose, acacia gum or xanthan gum.

The cellulose polymer used in the tablet coating composition may be selected from the group consisting of: methylcellulose, hydroxypropylcellulose (HPC), hydroxypropylmethylcellulose (HPMC or hypromellose), hydroxyethylcellulose (HEC), hydroxyethylmethylcellulose (HEMC), and a combination of any two or more of the aforementioned. In some embodiments, the cellulose polymer is hydroxypropylmethylcellulose. Hydroxypropylmethylcellulose is available in a range of viscosities. The viscosity of the hydroxypropylmethylcellulose that is used may depend on the specific application. Hydroxypropylmethylcellulose having a viscosity of 4.5 centipoise (cps), 5 cps, 6 cps, 15 cps, or even 50 cps may be suitable. In some embodiments, the viscosity of the hydroxypropylmethylcellulose is about 4 cps to about 6 cps. In some embodiments, the hydroxypropylmethylcellulose has a viscosity of 4.5 cps. In some embodiments, the hydroxypropylmethylcellulose has a viscosity of 5 cps. In some embodiments, the hydroxypropylmethylcellulose has a viscosity of 6 cps.

The plasticiser used in the tablet coating composition may be a polyethylene glycol. The polyethylene glycol may have a molecular weight of about 4000 to about 20000. In some embodiments, the polyethylene glycol has a molecular weight of about 6000.

The sweetener used in the tablet coating composition may be a sweetener that has a sweetness greater than the sweetness of sucrose. This may be any suitable natural or artificial sweetener having the requisite sweetness. In some embodiments, the sweetener is sucralose.

The tablet coating composition may include, by dry weight of the tablet coating composition, 40-80% cellulose polymer, 5-30% plasticiser, 0.1-5% sweetener, and 5-33% powdered flavour composition. In some embodiments, the tablet coating composition also includes, by dry weight of the tablet coating composition, 5-25% of pigments. In some embodiments, the tablet coating composition includes, by dry weight of the tablet coating composition, 40-60% cellulose polymer, 10-30% plasticiser, 0.1-2% sweetener, and 10-30% powdered flavour composition.

The tablet coating composition may also contain other components including, but not limited to, adherents, lubricants, emulsifiers, anti-foaming agents, colourants, coating polymers, fragrances, and active agents.

In some embodiments, the tablet coating composition is dissolved or suspended in a liquid so that it can be applied to a tablet. Thus, the present invention further provides a tablet coating fluid including the aforementioned tablet coating composition and a liquid.

In some embodiments, the liquid of the tablet coating fluid is a solvent. The solvent may be an organic solvent, an aqueous solvent or water. In some embodiments, the solvent is an aqueous solvent containing ethanol and water. In some embodiments, the solvent is about 20% to about 80% ethanol/water. In some embodiments, the solvent is 60% ethanol/water.

In some embodiments, the coating fluid includes, by weight, 6-7% cellulose polymer, 1-2% plasticiser, 0.1-0.3% sweetener, 1-3% powdered flavour composition, 52-53% ethanol, and 35-36% water. In some embodiments, the coating fluid also includes 1-2% of pigments. In some embodiments, the coating fluid includes 6-7% hypromellose 5 or 6 cps, 1-2% polyethylene glycol 6000 (plasticiser), 1-2% talc-purified/titanium dioxide/colour, 1-3% flavour/maltodextrin powder (the weaker the flavour the more is needed), 0.1-0.2% sucralose (depending on how sweet consumers like it), about 53% ethanol 96% BP, and about 35% water (purified).

The present invention also provides a pharmaceutical tablet including:
a core containing an active agent; and
a coating,
   the coating formed from a tablet coating composition including a cellulose polymer, a plasticiser, a sweetener, and a powdered flavour composition, the powdered flavour composition including a flavourant associated with a solid carrier.

The cellulose polymer, plasticiser, sweetener and powdered flavour composition may be as described earlier.

The active agent may be a pharmaceutically active agent, a nutraceutically active agent or a veterinarally active agent.

In some embodiments, the coating is applied to the core as a fluid by spray coating. The coating may be about 1% to about 6% by weight of the total weight of the tablet.

The present invention also provides a process for preparing a coated tablet, the process including:
combining a cellulose polymer, a plasticiser, a sweetener, a powdered flavour composition and a liquid to form a tablet coating fluid, the powdered flavour composition including a flavourant associated with a solid carrier;
applying the tablet coating fluid to a core containing an active agent; and
removing a majority of the liquid to provide a coated tablet.

The present invention also provides a process for preparing a coated tablet, the process including:
providing a tablet coating composition including a cellulose polymer, a plasticiser, a sweetener, and a powdered flavour composition, the powdered flavour composition including a flavourant associated with a solid carrier;
combining the tablet coating composition and a liquid to form a tablet coating fluid;
applying the tablet coating fluid to a core containing an active agent; and removing a majority of the fluid to provide a coated tablet.

The present invention also provides a process for preparing a coated tablet, the process including:
providing a tablet coating fluid, the tablet coating fluid formed from a tablet coating composition including a cellulose polymer, a plasticiser, a sweetener, and a powdered flavour composition, the powdered flavour composition including a flavourant associated with a solid carrier, and a liquid;
applying the tablet coating fluid to a core containing an active agent; and
removing a majority of the liquid to provide a coated tablet.

The present invention also provides for use of a powdered flavour composition in the preparation of tablet coating composition, the tablet coating composition including a cellulose polymer, a plasticiser, a sweetener, and a powdered flavour composition, wherein the powdered flavour composition includes a flavourant associated with a solid carrier.

The present invention also provides for use of a tablet coating composition as described herein in the preparation of a coated tablet for the treatment of a disease, condition or disorder in a human or animal.

The present invention also provides for a method of treating a disease, condition or disorder in a human or animal, the method including administering to the human or animal a coated tablet as described herein, wherein the active agent is suitable for the treatment of the disease, condition or disorder.

### DETAILED DESCRIPTION

Before proceeding to describe the present invention, and embodiments thereof, in more detail it is important to note that various terms that will be used throughout the specification have meanings that will be well understood by a skilled addressee. However, for ease of reference, some of these terms will now be defined.

The term “active agent”, and variations thereof, as used herein means a substance or group of substances that illicit a physiological response when administered to a human or animal. The term includes a substance or group of substances that is intended for use in the diagnosis, cure, mitigation, treatment or prevention of an undesirable state in a human or animal. The active agent may be a pharmaceutically active agent, a nutraceutically active agent or a veterinarally active agent. For example, the active agent may be a drug that is used therapeutically to treat or prevent a disease state in humans or animals. Examples of active agents include pharmaceutical actives, therapeutic actives, vitamins, minerals, nutritional supplements, dietary supplements, cosmetic actives, veterinary actives, nutraceuticals, growth regulators, sterilants, pheromones, nutrients, proteinaceous materials, genes, chromosomes, DNA and other biological materials.

The terms “active pharmaceutical agent”, “pharmaceutically active agent”, “active drug” and “drug” as used herein mean any active pharmaceutical ingredient (“API”), including its pharmaceutically acceptable salts, as well as in the anhydrous, hydrated, and solvated forms, in the form of prodrugs, and in the individually optically active enantiomers of the API as well as polymorphs of the API.

The terms “active nutraceutical agent” and “nutraceutically active agent” as used herein mean any food or nutrient-based substance that may provide medicinal or health benefits, including the prevention and treatment of disease.

The term “pharmaceutically acceptable” as used herein means a substance or composition that is compatible chemically and/or toxicologically with the other ingredients including a formulation, and/or the mammal being treated.

The term “tablet” as used herein means a single dosage form, i.e. the single entity containing the active agent that is administered to the subject. The term “tablet” also includes a tablet that may be a combination of one or more “minitablets” or “cores”. The mintablets or cores may be used in capsules or even sachets (if smaller than about 3 mm).

The term “pharmaceutical tablet” as used herein means a tablet that contains one or more active agents, and includes a tablet for pharmaceutical, nutraceutical or veterinary use.

The term “core” as used herein means any structure that is surrounded (partially or wholly) by a wall, membrane, or coating. The wall, membrane, or coating can be a functional or non-functional coating.

The terms “powder” and “powdered” as used herein mean a particulate material consisting of a loose aggregation of fine solid particles.

The terms “treating”, “treat”, or “treatment” refer generally to amelioration or elimination of a disease, condition or disorder once it has been established. The term “prophylaxis” refers generally to treatment to prevent the onset of a disease, condition or disorder or of a process that can lead to the disease, condition or disorder (“primary” prophylaxis), or the recurrence of symptoms of a disease, condition or disorder.

The terms “effective amount” and “therapeutically effective amount” refer generally to an amount of a compound or composition that (i) treats or prevents the particular disease, condition, or disorder, (ii) attenuates, ameliorates, or eliminates one or more symptoms of the particular disease, condition, or disorder, or (iii) prevents or delays the onset of one or more symptoms of the particular disease, condition, or disorder.

The terms “subject” and “patient” as used herein mean all members of the animal kingdom, including humans.

The present invention provides a tablet coating composition including a cellulose polymer, a plasticiser, a sweetener, and a powdered flavour composition. The powdered flavour composition includes a flavourant associated with a solid carrier.

The powdered flavour composition may be prepared by spray drying the flavourant with the carrier, thereby providing a flavour composition in which the flavourant at least partially coats, or is associated with, some of the granules or particles of the carrier.

The flavourant may be any natural, artificial or synthetic compound or mixture of compounds that is pharmaceutically, nutraceutically or veterinarally acceptable. An illustrative list of flavourants for pharmaceutical and nutraceutical applications includes volatile oils, synthetic flavour oils, flavouring aromatics, oils, liquids, oleoresins and extracts derived from plants, leaves, flowers, fruits, stems, roots, and combinations thereof. Non-limiting examples of flavour oils include spearmint oil, cinnamon oil, oil of wintergreen (methyl salicylate), peppermint oil, clove oil, bay oil, anise oil, eucalyptus oil, thyme oil, cedar leaf oil, oil of nutmeg, allspice, oil of sage, mace, oil of bitter almonds, cassia oil, and combinations thereof. Suitable flavourants also include, for example, artificial, natural and synthetic fruit flavours such as citrus oils (e.g., lemon, orange, lime, and grapefruit), fruit essences (e.g., apple, pear, peach, grape, strawberry, raspberry, cherry, plum, pineapple, apricot or other fruit flavours). Other useful artificial, natural and synthetic flavourants include chocolate, coffee, vanilla, honey powders, and combinations thereof. Other useful flavourants include aldehydes and esters, such as benzaldehyde (cherry, almond), citral (lemon, lime), neral (lemon, lime), decanal (orange, lemon), aldehyde C-8 (citrus fruits), aldehyde C-9 (citrus fruits), aldehyde C-12 (citrus fruits), tolyl aldehyde (cherry, almond), 2,6-dimethyloctanal (green fruit), 2-dodenal (citrus mandarin), and combinations thereof.

An illustrative list of flavourants for veterinary applications includes volatile oils, synthetic flavour oils, flavouring aromatics, oils, liquids, oleoresins and extracts derived from animals, plants, leaves, flowers, fruits, stems, roots, and combinations thereof. Non-limiting examples of flavourants include meat extract, fish extract, and vegetable extract.

The carrier may include a dextrin. In some embodiments, the dextrin is maltodextrin. Maltodextrin is a polysaccharide that is widely used as a food additive and pharmaceutical excipient and is widely available commercially. The maltodextrin ideally has a Dextrose Equivalent (DE) of about 15 to about 20. As the DE of the maltodextrin increases, so does sweetness and solubility. However, materials having a DE of greater than about 20 are corn syrup solids and dextrose which are more hygroscopic. Dextrose is also less favoured by diabetics and consumers may also prefer “sugarless” products having fewer calories.

The powdered flavour composition may contain flavourant in an amount from about 1% to about 20%, by weight, with the remainder being carrier and, optionally, other components. In some embodiments, the powdered flavour composition contains about 10%, by weight, of the flavourant and about 90%, by weight, maltodextrin.

As discussed, the powdered flavour composition may contain other components in addition to the carrier. For example, the powdered flavour composition may contain another saccharide or polysaccharide, such as glucose or dextrose. Alternatively, or in addition, the powdered flavour composition may contain a sweetener, such as a natural or artificial sweetener. Alternatively, or in addition, the powdered flavour composition may contain a gum, such as sodium carboxymethylcellulose, acacia gum or xanthan gum (e.g. Keltrol F). Gums can be added to improve mouthfeel as they rapidly swell when put in the mouth and quickly release the flavour from the coating by breaking it up.

Benefits of using the powdered flavour composition and subsequently combining it with other components to form the tablet coating composition is that it becomes both a flavour and a functional ingredient, there are less ingredients to weigh out, and the powdered flavour composition is normally less volatile than if a liquid flavourant is used, which is useful for stability and manufacturability. Indeed, the volatility of liquid flavourants makes accurate dosing of the flavourant difficult under normal manufacturing conditions. Also, the solvent (or other volatile component(s)) of liquid flavourants can have detrimental effects on the tablet core when the tablets are coated because the flavour can tend to leach through to the tablet. In addition, we have found that when making a coating fluid containing the powdered flavour composition the viscosity of the coating fluid is greater than if a liquid flavourant is used and the film forming behaviour of the coating fluid is improved. Including a powdered flavour composition provides a film with good strength and adhesion.

Surprisingly, we have found that the use of a powdered flavour composition in formulating the tablet coating composition gives rise to a coated tablet that may have improved gloss and/or mouthfeel compared to a coated tablet formed from a coating composition in which a liquid flavourant is used. Furthermore, the carrier that is used in the tablet coating composition may provide for improved gloss and mouthfeel in the final product.

We have found that the higher the percentage of powdered flavour composition used in the tablet coating composition, the more slippery the coating feels and the more glossy it becomes. However, if too much powdered flavour composition is used the coating can appear frosty (whiter coats will hide this).

The powdered flavour composition is combined with the cellulose polymer, the plasticiser, and the sweetener to form the tablet coating composition. The tablet coating composition is suitable for coating a tablet to produce a coated tablet that has a pleasant taste and mouthfeel and is easy to swallow.

The tablet to be coated may contain one or more of any active agent. Indeed, active agents which may be effectively coated are not limited and include pharmaceutically active agents, nutraceutically active agents and veterinarally active agents, such as those typically delivered in a tablet dosage form. The flavoured coating composition is particularly suitable for coating tablets containing unpleasant tasting pharmaceutically active agents, nutraceutically active agents or veterinarally active agents. Examples include, but are not limited to: analgesics and antiphlogistics such as aspirin, acetaminophen, phenacetin; steroids including antinflammatory steroids; enzymes, proteins, antibiotics or antimycrophotics including penicillin and its derivatives; anesthetics, vasodiolators such as nitroglycerin, anticarcinogens, sulfonamide drugs, sedatives, tranquilizing and hypnotic agents, bronchial-dilating agents, potassium chloride, mixtures thereof, and the like.

Pharmaceutically or veterinarally active agents can include, for example, medicaments or drugs, e.g., analgesics, anti-inflammatory agents, anthelmintics, anti-arrhythmic agents, antibiotics, anticoagulants, antidepressants, antidiabetic agents, antidiarrheal agents, antiemetic agents, antiepileptics, antihistamines, antihypertensive agents, antimuscarinic agents, antimycobacterial agents, antineoplastic agents, immunosuppressants, antithyroid agents, anti-tussive agents, antiviral agents, anxiolytic sedatives, astringents, beta-adrenoceptor blocking agents, cardiac ionotropic agents, corticosteroids, cough suppressants, diagnostic agents, diagnostic imaging agents, diuretics, dopaminergics, haemostatics, immunological agents, lipid regulating agents, muscle relaxants, parasympathomimetics, parathyroid calcitonin and biphosphonates, prostaglandins, radio-pharmaceuticals, steroids, anti-allergic agents, stimulants and anoretics, sympathomimetics, thyroid agents, vasodilators, xanthines, and combinations thereof.

Nutraceutically active agents can include, for example, dietary supplements, minerals, vitamins, and the like, and combinations thereof. Examples of nutraceutically active agents include, vitamin A, vitamin D, vitamin E (e.g., d-alpha-tocopherol, d-alpha-tocopheryl acetate, dl-alpha-tocopherol and dl-alpha-tocopheryl acetate), vitamin B1 and derivatives thereof, vitamin B2 and derivatives thereof, vitamin B6 and derivatives thereof (e.g., pyridoxine hydrochloride), vitamin C and derivatives thereof (e.g., ascorbic acid, sodium L-ascorbate, etc.), vitamin B12 and derivatives thereof, fluoride (e.g., sodium fluoride), calcium, magnesium, iron, proteins, amino acids, amino saccharides (amino sugars), oligosaccharides, and combinations thereof. There may be circumstances in which a pharmaceutically active agent may also function as a nutraceutically active agent, and in which a nutraceutically active agent may also function as a pharmaceutically active agent.

The tablet may contain the active agent(s) on its own or, more commonly, the active agent admixed with one or more tabletting excipients, carriers, binders and the like. To produce tablets, particles containing the active agent may be mixed or blended with the desired excipient(s), if any, using conventional procedures and the resulting mixture compressed according to conventional tabletting procedure using a suitably sized tabletting tool.

The tablet coating composition contains the powdered flavour composition, the cellulose polymer, the plasticiser, the sweetener, and, optionally, other pharmaceutically acceptable excipients. The tablet coating composition typically includes, by dry weight of the tablet coating composition, 40-80% cellulose polymer, 5-30% plasticiser, 0.1-5% sweetener, and 5-33% powdered flavour composition. In some embodiments, the tablet coating composition also includes, by dry weight of the tablet coating composition, 5-25% pigments. In some embodiments, the tablet coating composition includes, by dry weight of the tablet coating composition, 40-60% cellulose polymer, 10-30% plasticiser, 0.1-2% sweetener, and 10-30% powdered flavour composition. In some embodiments, the tablet coating composition includes, by dry weight of the tablet coating composition, about 52% cellulose polymer, about 13% plasticiser, about 0.5% sweetener, and about 21% flavour composition, the remainder being pigments.

The cellulose polymer may be any film-forming cellulose polymer. For example, the cellulose polymer may be selected from the group consisting of: methylcellulose, hydroxypropylcellulose (HPC), hydroxypropylmethylcellulose (HPMC or hypromellose), hydroxyethylcellulose (HEC), hydroxyethylmethylcellulose (HEMC), and a combination of any two or more of the aforementioned. In some embodiments, the cellulose polymer is hydroxypropylmethylcellulose (HPMC). Suitable HPMC include those having a viscosity from about 1 to about 100 centipoise (cps), in particular from about 3 to about 15 cps. HPMC having a low viscosity, i.e. from about 4 to about 6 cps, is useful. HPMC having a viscosity of 4.5, 5 or 6 cps is commercially available.

The plasticiser may be any substance that improves the plastic properties of the coating when formed. For example, the plasticiser may be selected from the group consisting of: glycerin, triethyl citrate, 1,2-propylene glycol, polyethylene glycol, and propylene glycol.

In some embodiments, the plasticiser is polyethylene glycol (also known as macrogol in the European pharmacopoeia). Polyethylene glycol (PEG) is a flexible, water soluble polymer of ethylene oxide. PEG polymers have different molecular weights and different physical properties (e.g. viscosity) due to chain length effects. High molecular weight PEG polymers are less hygroscopic and less likely to leach into the tablet than some other lower molecular weight plasticisers. The PEG used in the tablet coating composition may have a molecular weight of about 4000 to about 20000 (i.e. PEG 4000 to PEG 20000). Specific PEGS include, but are not limited to, PEG 6000 and PEG 8000. In some embodiments, the polyethylene glycol has a molecular weight of about 6000 (i.e. PEG 6000). The PEG 6000 that is commercially available as Carbowax™, Lutrol™ or PolyGlicol™6000 PF is suitable for use in the tablet coating composition.

The sweetener used in the tablet coating composition is typically a sweetener that has a sweetness greater than the sweetness of sucrose. In other words, the sweetness of the sweetener may be greater than 1.0 relative to the sweetness of sucrose. Examples of sweeteners that may be used in the tablet coating composition include, but are not limited to: saccharin and its various salts, such as sodium salt; dipeptide sweeteners such as aspartame and alitame; dihydrochalcone compounds, glycyrrhizin; extracts of Stevia Rebaudiana (Stevia); chloro derivatives of sucrose such as sucralose; synthetic sweeteners such as 3,6-dihydro-6-methyl-1-1-1,2,3-oxathiazin-4-1-2,2-dioxide, particularly the potassium salt (acesulfame-K), and sodium and calcium salts thereof; neohesperidin, thaumatin and cyclamate. The sweetener may be a single sweetener or a combination of sweeteners. Sucralose is particularly suitable for use in the tablet coating composition.

Optionally, the tablet coating composition includes one or more pharmaceutically acceptable excipients, such as adherents, lubricants, emulsifiers, anti-foaming agents, colourants, coating polymers, fragrances, active agents, and the like.

In some embodiments, the tablet coating composition contains one or more colourants. Suitable colourants include colours, dyes, lakes, and pigments. Examples include, but are not limited to, talc, titanium dioxide, iron oxides, FD&C and D&C lakes, magnesium carbonate, pyrogenic silica, channel black, insoluble dyes, and mixtures of any two or more thereof. The colourant could also be a natural colour, such as riboflavin, carmine 40, cochineal, curcumin, annatto, and mixtures thereof. The colour or combination of colours may be selected by those of skill in the art based upon a need at the time of the coating operation. In the absence of a colourant, the tablet coating composition may produce a frosted coating on a coated tablet, though a frosted coating tends to be less noticeable with a white/paler background.

A coated tablet is formed by forming a tablet coating fluid from the powdered tablet coating composition in a suitable liquid and applying the tablet coating fluid to tablets. A majority of the liquid is then removed to provide the coated tablet. The liquid may be a solvent in which the components of the tablet coating composition are soluble so as to form a tablet coating fluid. Alternatively, the liquid may be a liquid in which some or all of the components of the tablet coating composition are either insoluble or partially soluble so as to form a tablet coating suspension.

The solvent may be an organic solvent, an aqueous solvent or water. In some embodiments, the solvent is an aqueous solvent containing ethanol and water. In some embodiments, the solvent is about 20% to about 80% ethanol/water. In some embodiments, the solvent is 60% ethanol/water. This solvent is particularly useful for coating moisture sensitive tablets at low temperatures.

In some embodiments, the tablet coating fluid includes, by weight, 6-7% cellulose polymer, 1-2% plasticiser, 0.1-0.3% sweetener, 1-3% powdered flavour composition, and about 88% liquid. In some embodiments, the liquid includes, by weight, about 53% ethanol, and about 35% water. In some embodiments, the coating fluid also includes 1-2% of pigments. In some specific embodiments, the coating fluid includes, by weight, 6-7% hypromellose 5 or 6 cps, 1-2% polyethylene glycol 6000 (plasticiser), 1-2% talc-purified/titanium dioxide/colour, 1-3% flavour/maltodextrin powder (the weaker the flavour the more is needed), 0.1-0.3% sucralose (depending on how sweet consumers like it), 53% ethanol 96% BP, and 35% water (purified).

The tablet coating composition can be applied to the tablets in a batch, semi-continuous or continuous process or some combination thereof in a manner which produces a satisfactory uniformly coated tablet. Various methods for coating tablets with solutions or suspensions of coating compositions are known, including rotating pan, fluid bed, spouted bed, coascervation tank, and pressing methods. In most coating methods, coating solutions are sprayed onto tablets as the tablets are being agitated in a pan, fluid bed, etc. As the fluid is being sprayed, a thin film is formed that adheres directly to each tablet. The coating may be formed by a single application or may be built up in layers through the use of multiple spraying cycles. A majority of the solvent is then removed, for example, by evaporating the solvent by passing air over the surface of the tumbling tablets. The skilled person will appreciate that not all of the solvent need be removed to provide a stable, coated tablet and, therefore, it is contemplated that a small percentage of the solvent may be “trapped” in the coating. However, a majority of the solvent is removed to provide a film or coating on the surface of the tablet.

Although in some embodiments the coating composition will initially be an hydroalcoholic composition, the tablet coating will typically be dried or substantially dried prior to, upon its exit or removal from the coating application system or at sometime in preparing coated tablets. The coated tablets may be placed in suitable packaging then if desired.

The amount of coating provided to the surface of the tablet is an effective amount and is typically that amount which provides a minimum effective coverage of the exterior surface area of the tablet although that may not necessarily always be the case and partial coverage of the exterior surface may also be suitable. In some embodiments, the amount of tablet coating composition which is coated onto tablets is that amount which provides a coated tablet having from about 1% to about 6% weight percent of the total tablet weight. In some embodiments in which the tablets are very small the coating may be up to about 30% weight percent of the total tablet weight.

The tablet coating composition may be coated onto tablets which are uncoated or tablets which have been coated with one or more prior coatings (overcoating). An initial coating may include one or more polymers such as cellulosics, dextrins, acrylics, any colours or other pharmaceutical coating material.

The coating could be formed on tablets which are placebos or blanks. The tablet may be any shape or size which allows the tablet to be effectively consumed by humans or animals. The tablet may be any tablet, particle, micronized particle, particulate, pellet, pill, core, powder, granule, granulate, small mass, seed, speck, sphere, crystal, bead, agglomerate, and mixtures thereof. Typically, the tablet will be in a form sufficiently stable physically and chemically to be effectively coated in a system which involves some movement of the tablet, as for example in a fluidised bed, such as in a fluidised bed dryer or perforated pan or accela—type coater.

The tablet coating composition can be used in the preparation of a coated tablet for the treatment of a disease, condition or disorder in a human or animal. Furthermore, the present invention provides for a method of treating a disease, condition or disorder in a human or animal, the method including administering to the human or animal coated tablet as described herein, wherein the active agent is suitable for the treatment of the disease, condition or disorder.

The coated tablets may be internally consumed by humans and animals in a customary manner. The amount of active agent administered will typically treat and reduce or alleviate a condition. A therapeutically effective amount can be readily determined by an attending diagnostician by the use of conventional techniques and by observing results obtained under analogous circumstances. In determining the therapeutically effective amount a number of factors are to be considered including but not limited to, the species of animal, its size, age and general health, the specific condition involved, the severity of the condition, the response of the patient to treatment, the particular compound administered, the mode of administration, the bioavailability of the preparation administered, the dose regime selected, the use of other medications and other relevant circumstances.

A preferred dosage will be a range from about 0.01 to 300 mg per kilogram of body weight per day. A suitable dose can be administered in multiple sub-doses per day.

Coated tablets of the present invention typically have one or more enhanced properties such as higher gloss, better mouthfeel, good coating adhesion, non-tackiness (slipperiness when wet), being swallowable with little or no accompanying liquid, better taste, and the like. Any of these properties may help people remember that they have taken the tablet, i.e. there is improved compliance.

Examples of materials and methods for use with the compositions and methods of the present invention will now be provided. In providing these examples, it is to be understood that the specific nature of the following description is not to limit the generality of the above description.

### Example 1

### Formation of a Coated Tablet Having a Vanilla Flavoured Coating

A tablet coating fluid was formed by combining the following ingredients.

| | | % | | |
|---|---|---|---|---|
| | | Weight | % Dry | |
| | | of | weight of | |
| | Amount | coating | coating | |
| Ingredient | (mg/tab) | fluid | composition | Function |
| Hypromellose 5 cps | 33 | 6.2% | 51.8% | Cellulose polymer |
| Polyethylene glycol 6000 | 8.25 | 1.5% | 13.0% | Plasticiser |
| Talc | 6.60 | 1.2% | 10.4% | Pigment (colourant) |
| Titanium dioxide | 2.20 | 0.4% | 3.5% | Pigment (colourant) |
| Ferric Oxide Yellow | 0.066 | 0.01% | 0.1% | Pigment (colourant) |
| Vanilla flavour/maltodextrin | 13.20 | 2.5% | 20.7% | Flavour composition |
| powder | | | | |
| Sucralose | 0.3300 | 0.1% | 0.5% | Sweetener |
| Ethanol 96% BP | 282.86 | 52.9% | — | Solvent |
| Water Purified | 188.57 | 35.2% | — | Solvent |
| TOTAL | 535.076 | 100.0% | 100% | |

The tablet coating fluid was then sprayed via a nozzle onto a bed of moving tablets. Typically exhaust temperatures of approximately 40 degrees Celsius are used. Typically a 3-4% coat weight is applied.

The example tablet composition was as follows:

| Ingredient | Amount | |
|---|---|---|
| Glucosamine Hydrochloride | 1500 | mg |
| Povidone | 78.95 | mg |
| Microcrystalline Cellulose | 200.05 | mg |
| Crospovidone | 5.00 | mg |
| Silicon dioxide | 3.00 | mg |
| Magnesium Stearate | 13.00 | mg |

We have found that a combination of maltodextrin (from powdered flavour)/hypromellose/polyethylene glycol/sucralose gives a good mouthfeel, taste, gloss and coating adhesion.

### Example 2

### Formation of a Coated Tablet Having a Very Sweet Berry Flavoured Coating

A coated tablet having a berry flavour was formed according to the methods described in Example 1 with a tablet coating fluid formed with the following ingredients.

| | | % | | |
|---|---|---|---|---|
| | | Weight | % Dry | |
| | | of | weight of | |
| | Amount | coating | coating | |
| Ingredient | (mg/tab) | fluid | composition | Function |
| Hypromellose 5 cps | 25 | 6.6% | 53.3% | Cellulose polymer |
| Polyethylene glycol 6000 | 6.25 | 1.6% | 13.3% | Plasticiser |
| Talc | 3.21 | 0.8% | 6.8% | Pigment (colourant) |
| Cochineal | 1.79 | 0.5% | 3.8% | Pigment (colourant) |
| Iron Oxide red | 0.06 | 0.02% | 0.1% | Pigment (colourant) |
| Titanium dioxide | 1.67 | 0.4% | 3.6% | Pigment (colourant) |
| Berry flavour/maltodextrin | 8.06 | 2.2% | 17.2% | Flavour composition |
| powder | | | | |
| Sucralose | 0.83 | 0.22% | 1.8% | Sweetener |
| Ethanol 96% BP | 200.00 | 52.6% | — | Solvent |
| Water Purified | 133.33 | 35.1% | — | Solvent |
| TOTAL | 380.20 | 100.0% | 100% | |

### Example 3

### Formation of a Coated Tablet Having a Berry Flavoured Coating

A coated tablet having a berry flavour was formed according to the methods described in Example 1 with a tablet coating fluid formed with the following ingredients. This coating has less flavour and is not as sweet as the coating of Example 2.

| | | % | | |
|---|---|---|---|---|
| | | Weight | % Dry | |
| | | of | weight of | |
| | Amount | coating | coating | |
| Ingredient | (mg/tab) | fluid | composition | Function |
| Hypromellose 5 cps or 6 cps | 35 | 6.6% | 57.2% | Cellulose polymer |
| Polyethylene glycol 6000 | 8.75 | 1.7% | 14.3% | Plasticiser |
| Talc - purified | 4.67 | 0.9% | 7.6% | Pigment (colourant) |
| Cochineal | 2.33 | 0.44% | 3.8% | Pigment (colourant) |
| Iron Oxide red | 0.125 | 0.067% | 0.2% | Pigment (colourant) |
| Titanium dioxide | 2.33 | 0.4% | 3.8% | Pigment (colourant) |
| Berry flavour/maltodextrin | 7.25 | 1.37% | 11.9% | Flavour composition |
| powder | | | | |
| Sucralose | 0.70 | 0.13% | 1.1% | Sweetener |
| Ethanol 96% BP | 280.00 | 53.0% | — | Solvent |
| Water Purified | 186.67 | 35.4% | — | Solvent |
| TOTAL | 527.825 | 100.0% | 100% | |

### Example 4

### Formation of a Coated Tablet Having a Citrus Flavoured Coating

A coated tablet having a citrus flavour was formed according to the methods described in Example 1 with a tablet coating fluid formed with the following ingredients.

| | | % | | |
|---|---|---|---|---|
| | | Weight | % Dry | |
| | | of | weight of | |
| | Amount | coating | coating | |
| Ingredient | (mg/tab) | fluid | composition | Function |
| Hypromellose 5 cps or 6 cps | 35 | 6.6% | 52.6% | Cellulose polymer |
| Polyethylene glycol 6000 | 8.75 | 1.7% | 13.1% | Plasticiser |
| Talc - purified | 5.60 | 1.1% | 8.4% | Pigment (colourant) |
| Iron oxide yellow | 1.00 | 0.19% | 1.5% | Pigment (colourant) |
| Iron oxide red | 0.196 | 0.04% | 0.3% | Pigment (colourant) |
| Titanium dioxide | 3.50 | 0.66% | 5.3% | Pigment (colourant) |
| Orange flavour/maltodextrin | 11.67 | 2.20% | 17.5% | Flavour composition |
| powder | | | | |
| Sucralose | 0.88 | 0.17% | 1.3% | Sweetener |
| Ethanol 96% BP | 280.00 | 53.0% | — | Solvent |
| Water Purified | 186.67 | 35.4% | — | Solvent |
| TOTAL | 533.266 | 100.0% | 100% | |

Finally, it will be appreciated that various modifications and variations of the methods and compositions of the invention described herein will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are apparent to those skilled in the art are intended to be within the scope of the present invention.

1. A tablet coating composition including a cellulose polymer, a plasticiser, a sweetener, and a powdered flavour composition, the powdered flavour composition including a flavourant associated with a solid carrier.

2. A tablet coating composition according to claim 1, wherein the cellulose polymer is selected from the group consisting of: methylcellulose, hydroxypropylcellulose (HPC), hydroxypropylmethylcellulose (HPMC or hypromellose), hydroxyethylcellulose (HEC), hydroxyethylmethylcellulose (HEMC), and a combination of any two or more of the aforementioned.

3. A tablet coating composition according to claim 2, wherein the cellulose polymer is hydroxypropylmethylcellulose.

4. A tablet coating composition according to claim 3, wherein the hydroxypropylmethylcellulose has a viscosity of about 4 to about 6 centipoise.

5. A tablet coating composition according to claim 4, wherein the hydroxypropylmethylcellulose has a viscosity selected from the group consisting of: 4.5 centipoise, 5 centipoise, and 6 centipoise.

6. A tablet coating composition according to claim 1 wherein the composition including about 40% to about 80% (by dry weight of the tablet coating composition) cellulose polymer.

7. A tablet coating composition according to claim 1 wherein the plasticiser is polyethylene glycol.

8. A tablet coating composition according to claim 7, wherein the polyethylene glycol has a molecular weight of about 6000.

9. A tablet coating composition according to claim 1 wherein the composition including about 5% to about 30% (by dry weight of the tablet coating composition) plasticiser.

10. A tablet coating composition according to claim 1 wherein the sweetener has a sweetness greater than the sweetness of sucrose.

11. A tablet coating composition according to claim 1 wherein the sweetener is selected from the group consisting of: saccharin and its salts; dipeptide sweeteners; dihydrochalcone compounds; glycyrrhizin; extracts of Stevia Rebaudiana (Stevia); chloro derivatives of sucrose; synthetic sweeteners; and cyclamate.

12. A tablet coating composition according to claim 11, wherein the sweetener is sucralose.

13. A tablet coating composition according to claim 1 wherein the composition including about 0.1% to about 5% (by dry weight of the tablet coating composition) sweetener.

14. A tablet coating composition according to claim 1 wherein the solid carrier includes a dextrin.

15. A tablet coating composition according to claim 14, wherein the dextrin is maltodextrin.

16. A tablet coating composition according to claim 15, wherein the maltodextrin has a Dextrose Equivalent of about 15 to about 20.

17. A tablet coating composition according to claim 1 wherein the flavourant is selected from one or more of the group consisting of: spearmint oil, cinnamon oil, oil of wintergreen (methyl salicylate), peppermint oil, clove oil, bay oil, anise oil, eucalyptus oil, thyme oil, cedar leaf oil, oil of nutmeg, allspice, oil of sage, mace, oil of bitter almonds, cassia oil, vanilla, citrus oils (e.g., lemon, orange, lime, and grapefruit), fruit essences (e.g., apple, pear, peach, grape, strawberry, raspberry, cherry, plum, pineapple, apricot or other fruit flavours), honey powders, benzaldehyde (cherry, almond), citral (lemon, lime), neral (lemon, lime), decanal (orange, lemon), aldehyde C-8 (citrus fruits), aldehyde C-9 (citrus fruits), aldehyde C-12 (citrus fruits), tolyl aldehyde (cherry, almond), 2,6-dimethyloctanal (green fruit), 2-dodenal (citrus mandarin).

18. A tablet coating composition according to claim 1 wherein the powdered flavour composition further includes a gum.

19. A tablet coating composition according to claim 18, wherein the gum is selected from one or more of the group consisting of: sodium carboxymethylcellulose, acacia gum, and xanthan gum.

20. A tablet coating composition according to claim 1 wherein the powdered flavour composition includes, by weight, about 1% to about 20% flavourant.

21. A tablet coating composition according to claim 1 wherein the composition including about 5% to about 33% (by dry weight of the tablet coating composition) powdered flavour composition.

22. A tablet coating composition according to claim 1 wherein the coating composition includes one or more other components selected from the group consisting of: adherents, lubricants, emulsifiers, anti-foaming agents, colourants, coating polymers, fragrances, and active agents.

23. A tablet coating composition according to claim 22, wherein the coating composition includes one or more colourants.

24. A tablet coating fluid including the tablet coating composition of claim 1 and a liquid.

25. A tablet coating fluid according to claim 24, wherein the liquid is a solvent.

26. A tablet coating fluid according to claim 25, wherein the solvent is an organic solvent, an aqueous solvent or water.

27. A tablet coating fluid according to claim 26, wherein the solvent is an aqueous solvent containing ethanol and water.

28. A tablet coating fluid according to claim 27, wherein the solvent is about 20% to about 80% ethanol/water.

29. A tablet coating fluid according to claim 28, wherein the solvent is 60% ethanol/water.

30. A pharmaceutical tablet including:
a core containing an active agent; and
a coating,
the coating formed from a tablet coating composition according to claim 1.

31. A pharmaceutical tablet according to claim 30, wherein the active agent is selected from the group consisting of: pharmaceutically active agents, nutraceutically active agents, veterinarally active agents, and combinations of any two or more of the aforementioned.

32. A pharmaceutical tablet according to claim 30 wherein the coating is 1 to 6% by weight of the total weight of the tablet.

33. A process for preparing a coated tablet, the process including:
combining a cellulose polymer, a plasticiser, a sweetener, a powdered flavour composition, and a liquid to form a tablet coating fluid, the powdered flavour composition including a flavourant associated with a solid carrier;
applying the tablet coating fluid to a core containing an active agent; and
removing a majority of the liquid to provide a coated tablet.

34. A process for preparing pharmaceutical tablets according to claim 30, including applying the tablet coating fluid to the core by spray coating.
